(19) United States
(12) Patent Application Publication
(10) Pub. No.: US 2024/0363218 A1
(43) Pub. Date: Oct. 31, 2024

(54) TREATMENT EVALUATION AND RECOMMENDATION USING IMPLANTABLE MAGNETOELECTRIC DEVICES

(71) Applicant: Motif Neurotech, Inc., Houston, TX (US)

(72) Inventors: Jacob Robinson, Houston, TX (US); Matthew Kuhn, Houston, TX (US)

(73) Assignee: Motif Neurotech, Inc., Houston, TX (US)

(21) Appl. No.: 18/636,096

(22) Filed: Apr. 15, 2024

Related U.S. Application Data
(60) Provisional application No. 63/462,775, filed on Apr. 28, 2023.

(51) Int. Cl. G16H 20/40; A61N 1/36; A61N 1/372; A61N 1/378; G16H 10/20; G16H 10/60; G16H 15/00; G16H 40/63

(52) U.S. Cl. CPC G16H 20/40; A61N 1/36139; A61N 1/3615; A61N 1/36171; A61N 1/37223; A61N 1/37264; A61N 1/37288; A61N 1/3787; G16H 10/20; G16H 10/60; G16H 15/00; G16H 40/63

(57) ABSTRACT

Embodiments disclosed herein generally relate to treatment evaluation and recommendation using implantable magnetoelectric devices. Particularly, aspects of the present disclosure are directed to a method that includes sending, to a user device of a subject, first software code that presents, at a user interface of the user device, requests for input that are informative of an instantaneous user characteristic of the subject. Second software code is sent to a base station that is coupled to implantable devices implanted in regions of the subject. The second software code causes the base station to send a signal to the implantable devices with an instruction to execute implant stimulation protocols. A communication that represents inputs detected at the user interface while or after the implant stimulation protocols were being executed is received. A treatment recommendation for the subject is determined based on the communication and is output.

### CROSS-REFERENCE OF RELATED APPLICATIONS

This application claims priority to U.S. Provisional Patent Application No. 63/462,775, filed on Apr. 28, 2023, titled “TREATMENT EVALUATION AND RECOMMENDATION USING IMPLANTABLE MAGNETOELECTRIC DEVICES”, which is incorporated by reference in its entirety for all purposes.

### FIELD

The present disclosure relates to implantable devices, and, in particular, to techniques for evaluation and recommending treatment using implantable magnetoelectric devices.

### BACKGROUND

Wireless implantable bioelectronics provide hope for revolutionary clinical therapies, such as treating neurological and psychiatric disorders by interfering directly with the nervous system. These devices can deliver controlled stimulation to modulate the electrical activities of the nervous system and/or record electrical, chemical, and physical properties for better diagnosis.

One challenge in the use of wireless bio-implants is reliably providing stimulation that reduces symptoms of a condition. While stimulation protocols may be defined for particular conditions, some subjects may not respond as expected to the developed stimulation protocol for their respective condition. In addition, due to the number of stimulation parameters and the various attributes of a subject that may impact how the subject responds to a stimulation protocol, it may be difficult to establish a stimulation protocol for effectively treating the condition of the subject. Accordingly, improvements for treatment using wireless bio-implants are desirable.

### SUMMARY

In some embodiments, a method is provided. A method can involve sending, to a user device of a subject, first software code that is configured to present, at a user interface of the user device, one or more requests for input that are informative of an instantaneous user characteristic of the subject, and sending, to a base station that is communicatively coupled to one or more implantable devices implanted in one or more regions of the subject, second software code that is configured to cause the base station to send a signal to the one or more implantable devices with an instruction to execute one or more implant stimulation protocols. The method can also involve receiving, from the user device and the base station, a communication that represents one or more inputs, wherein the one or more inputs were detected at the user interface while or after the one or more implant stimulation protocols were being executed, determining a treatment recommendation for the subject based on the communication, and outputting the treatment recommendation for the subject.

In some embodiments, at least one of the one or more implantable devices can include a magnetoelectric film, an electrical circuit coupled to the magnetoelectric film, and one or more electrodes.

In some embodiments, the base station can include a magnetic field generator and a magnetic transceiver.

In some embodiments, the method can further involve receiving an additional communication from one or more sensors that are physically or wirelessly connected to the base station, or from the one or more implantable devices during the execution of the first software code and the second software code and determining the treatment recommendation for the subject based on the additional communication.

In some embodiments, the method can further involve generating, by the base station, an additional communication by recording one or more stimulation times at which the one or more implantable devices deliver stimulation to the one or more regions, predicting, based on the communication and the additional communication, whether or a degree to which the one or more implant stimulation protocols are resulting in a target effect for the instantaneous user characteristic, and modifying the second software code based on the prediction.

In some embodiments, modifying the second software code can include determining a subset of the one or more implantable devices that are associated with delivering stimulation that results in the target effect and modifying the second software code to cause the subset of the one or more implantable devices to deliver stimulation while remaining implantable devices are inactive.

In some embodiments, the method can further involve generating, by the base station, an additional communication by recording one or more stimulation times at which the one or more implantable devices deliver stimulation to the one or more regions, predicting, based on the communication and the additional communication, whether or a degree to which the one or more implant stimulation protocols are resulting in a target effect for the instantaneous user characteristic, and determining the treatment recommendation based on the prediction.

In some embodiments, determining the treatment recommendation can include generating, by the base station, an additional communication by recording one or more stimulation times at which the one or more implantable devices deliver stimulation to the one or more regions, determining, based on the communication and the additional communication, a subset of the one or more implantable devices that are associated with delivering stimulation that results in a target effect for the instantaneous user characteristic, and generating the treatment recommendation to cause the subset of the one or more implantable devices to deliver stimulation while remaining implantable devices are inactive.

In some embodiments, the method can further involve accessing subject data of the subject, determining a predicted condition for the subject based on the subject data, and selecting the first software code and the second software code based on the predicted condition.

In some embodiments, the method can further involve receiving an additional communication from an eye tracking device and/or a facial recognition device during the execution of the first software code and the second software code and determining the treatment recommendation for the subject based on the additional communication.

In some embodiments, determining the treatment recommendation can include inputting the communication into a machine-learning model.

In some embodiments, the treatment recommendation comprises a modification to an amplitude, a frequency, and/or a timing of stimulation delivered by the one or more implantable devices.

Some embodiments of the present disclosure include a system including one or more data processors. The system can further include a non-transitory computer readable storage medium containing instructions which, when executed on the one or more data processors, cause the one or more data processors to perform part or all of one or more of the methods disclosed herein.

In some embodiments, a computer-program product is provided that is tangibly embodied in a non-transitory machine-readable storage medium. The computer-program product can include instructions configured to cause one or more data processors to perform part or all of one or more of the methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an exemplary system including an implantable device and a base station according to some aspects of the present invention;

FIG. 2 illustrates an exemplary computing environment for predicting a treatment recommendation using implantable devices magnetoelectric devices according to some aspects of the present invention; and

FIG. 3 illustrates a block diagram of a process for treatment evaluation and recommendation using implantable magnetoelectric devices according to some aspects of the present invention.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

While certain embodiments are described, these embodiments are presented by way of example only, and are not intended to limit the scope of protection. The apparatuses, methods, and systems described herein may be embodied in a variety of other forms. Furthermore, various omissions, substitutions, and changes in the form of the example methods and systems described herein may be made without departing from the scope of protection.

Implantable devices can be used to provide stimulation to the brain, or other nervous system regions, of subjects. The stimulations may be used (for example) to treat a medical condition (e.g., a neurological or psychiatric disorder) or a symptom. Typically, treatment decisions for providing stimulation using implantable devices are made through trial-and-error between a clinician and a subject to obtain stimulation parameters that result in a target effect (e.g., a reduction in the symptom or expression of the condition). In addition, stimulation parameters that result in the target effect for one subject with a condition may not result in the target effect for another subject with the same condition. So, determining stimulation parameters through trial-and-error for a subject may be inefficient and/or ineffective.

Embodiments of the present disclosure utilize magnetoelectric power for wirelessly powering mm-sized devices implanted deep inside the body by converting low-frequency (e.g., hundreds of kHz) magnetic fields to electric voltage using magnetoelectric transducers. Systems and methods that use magnetoelectric power results in several advantages (e.g., relative to RF, inductive coupling, ultrasound, and light power transfer techniques) including high power transmission efficiency with miniaturized size, high power delivery (e.g., greater than 1 mW), and high misalignment tolerance (e.g., angle offset and lateral offset) relative to inductive coupling and ultrasound power transfer devices. These features are empowered by using magnetoelectric materials that have sufficient power density, mechanical resonance frequency, and permeability to concentrate magnetic flux inside the material. Stimulation parameters can be delivered to a subject using these implantable devices during an evaluation period prior to treating the subject using stimulation. Accordingly, the subject's response to the stimulation parameters can be analyzed to determine a treatment recommendation for the subject.

In some embodiments, subject data (e.g., including demographic data, medical history data, sleep data, physical activity data, physiological data, and/or mood data) for a subject is accessed. Based on the subject data, a predicted condition for the subject can be determined. For instance, the subject may be predicted as having anxiety, depression, posttraumatic stress disorder, attention deficit hyperactivity disorder, Parkinson's, etc. Software code for execution by a user device of the subject and one or more implantable devices implanted in one or more target regions of the subject can then be selected based on the predicted condition. First software code that is sent to the user device can present a request for input at a user interface. The presentation may be task requesting input that is informative of an instantaneous user characteristic of the subject. For instance, if the subject is predicted to have depression, the first software code may cause a presentation of a task that is associated with evaluating a mood of the subject. Second software code can be sent to a base station that sends a signal to the implantable devices with an instruction to execute various implant stimulation protocols while the presentation occurs at the user device. During the execution of the first software code and the second software code, a communication that represents the input can be received at the base station or another device. A treatment recommendation for the subject can be determined based on the communication. The treatment recommendation for the subject can be output so that stimulation in accordance with the treatment recommendation can be delivered to the subject to treat the predicted condition. Accordingly, the present disclosure provides a controlled environment for evaluating implant stimulation protocols for a short period of time to evaluate their efficacy before deploying a treatment stimulation protocol for the subject. As such, the treatment recommendations may result in improved treatment outcomes for subjects.

### II. Example Systems

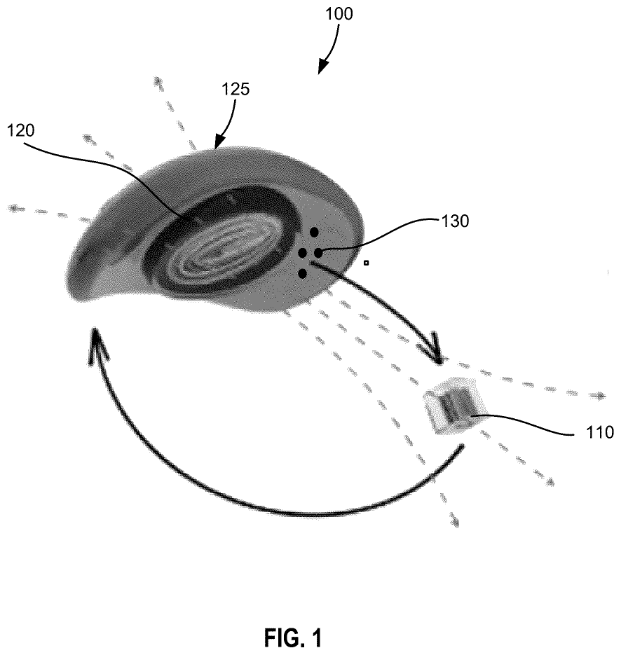

FIG. 1 illustrates an exemplary system 100 including an implantable device 110 and a base station 120 according to some aspects of the present invention. Using magnetoelectric communication, a magnetic field generator of the base station 120 can modulate a magnetic field applied to the implantable device 110. Modulating the electric load leads to magnetoelectric resonance frequency changes for frequency-shift-keying (FSK) modulation. A resonant frequency modulator of an electrical circuit of the implantable device 110 can implement capacitive load shifting to maximize the resonance frequency change without substantial fluctuations of received voltage. Magnetostriction effects can be leveraged for uplink communication using magnetoelectric backscatter received by a magnetic transceiver of the base station 120. In other cases, the uplink communication may be achieved with radio frequency (RF), near field communication (NFC), Bluetooth, or optical communication.

The magnetoelectric film of the implantable device 110 can be fabricated using a bilayer or trilayer sheet including one or more magnetostrictive layers and one or more piezoelectric layers. In an example, the magnetostrictive layer can use 27-μm Metglas, and the piezoelectric layer can use 254-μm-thick lead zirconium titanate PZT-5 material. When the magnetic field generator applies an external magnetic field, mechanical vibrations are generated in the magnetostrictive layer due to the Joule effect. Since the magnetostrictive layer and the piezoelectric layer are mechanically coupled, these vibrations are transferred to the piezoelectric layer to create an electric potential across the magnetoelectric film due to the direct piezoelectric effect.

The vibrations generated in the magnetostrictive layer result in a change in the material magnetization due to the Villari effect. This change can be seen as a backscattered field generated by the magnetoelectric film. Hence, utilizing these echoes as backscattered signals can enable uplink data transfer from the implantable device 110 to the base station 120. During the on-time of the applied magnetic field, the magnetoelectric materials vibrate at the applied field frequency. Since the generated backscattered field may oscillate at the same excitation frequency, it may be challenging to decouple the applied field frequency signal from the backscattered signal. To isolate the response of the magnetoelectric film, the backscattered field can be measuring during a ring-down period when the excitation field is off. Over the ring-down period, the magnetoelectric film dissipates the stored mechanical energy in the form of decaying voltage at its mechanical resonance frequency irrespective of the excitation field frequency.

In some examples, characteristics of the backscattered field can be changed at the implantable device 110 to modulate the backscattered signal for transmitting data. Although the backscattered field is generated by the magnetostrictive layer, modulating the characteristics of either the piezoelectric layer, magnetostrictive layer, or both can affect the backscattered field due to the mechanical coupling of the layers. The modulation of the backscattered field can be done by controlling the effective mechanical, magnetic, or electric properties of the composite. In an example, the magnetostrictive materials can be coated with a thin layer of stimuli-responsive polymers that respond to external stimuli by shifting the overall mass and mechanically modulating the resonance frequency of the magnetoelectric film. The resonant frequency modulator can modulate the resonant frequency of the magnetoelectric film by applying different electrical loading conditions that change a property of the magnetoelectric film. In some instances, an electric loading modulation technique can be used to tune the characteristics of the backscattered field. The electric load can be either an active load (e.g., a direct current (DC) biasing voltage) or a passive load (e.g., inductive, resistive, capacitive, or a combination thereof). Using passive loads may enable a miniaturized footprint of the implantable device 110 and limit constraints on the power budget.

Changing the capacitive or resistive loads across the magnetoelectric film can change the voltage across the magnetic film as well as its resonance frequency. Consequently, the amplitude and frequency of the backscattered field during the ring-down period are changed. Since a frequency shift may be more immune to the depth variation and misalignment that often occur during implantation of an implantable device, frequency modulation can be used to encode uplink data of the implantable device 110.

Capacitive load-shift-keying (LSK) modulation can result in a large frequency shift and small voltage drop to help resolve the frequency difference at the base station 120 and to improve the signal-to-noise ratio (SNR). The LSK-induced frequency-shift keying (FSK) can be used to digitally encode the data by switching between two load conditions: an open circuit and a capacitive load. To determine the suitable capacitive load to be implemented on the ASIC chip, the resonance frequency of the magnetoelectric film can be measured while connected in parallel to different capacitive loads and compared with that a mathematical model for validation.

In FIG. 1, the base station 120 is part of a wearable component 125 that also includes sensors 130 for communicating with the implantable device. The wearable component 125 is illustrated in FIG. 1 as being a headband that can be worn on a head of a user. In such examples, the implantable device 110 can be implanted in a brain of the user. For instance, the implantable device 110 may be implanted between 1 and 6 cm deep in the brain in a target region. The target region may be based on a condition or disorder of the user. For instance, the target region associated with providing stimulation to treat depression may be a different target region than is associated with providing stimulation to treat Parkinson's. One session of a therapeutic pulse sequence can range from 1 minute to 120 minutes of therapy consisting of continuous stimulation at a fixed frequency (e.g., 10 Hz or 130 Hz) a continuous series of bursts (e.g., continuous theta burst stimulation (cTBS): three pulses at 50 Hz repeated every 200 ms), or a pattern of bursts (e.g., intermittent theta burst stimulation (ITBS): three pulses at 50 Hz repeated every 200 ms for 2 seconds, repeated every 10 seconds). In all cases, the number of therapeutic sessions can be repeated multiple times per day with a time between therapies ranging from a few minutes to several hours. For the treatment of depression, the target region may be a dorsolateral prefrontal cortex, for the treatment of Parkinson's, the target region may be the bilateral subthalamic nucleus or the motor cortex, and for the treatment of posttraumatic stress disorder, the target region may be the anterior cingulate cortex.

The base station 120 can include electronics for controlling and monitoring stimulation provided to the user by the implantable device 110. The base station 120 may power and communicate with the implantable device 110 using magnetoelectric techniques, as previously described herein. The implantable device 110 can execute implant stimulation protocols based on instructions received from the electronics. An implant stimulation protocol can indicate an amplitude (e.g., between 0.2 and 3.5 V), shape (e.g., bi-phasic or mono-phasic), pulse width (e.g., between 0.05 and 1.5 ms), and frequency (e.g., between 0 and 200 Hz) for the stimulation. The electronics can then record stimulation times at which the implantable device 110 delivers the stimulation to the target tissue.

In some examples, multiple implantable devices may be implanted into the brain of the user. Each implantable device may be associated with a different implant stimulation protocol. So, a first implantable device may execute a first implant stimulation protocol of an amplitude of 1.5 V with a mono-phasic shape and a pulse width of 0.5 ms at a frequency of 50 Hz, while a second implantable device executes a second implant stimulation protocol of an amplitude of 1.5 V with a mono-phasic shape and a pulse width of 0.5 ms at a frequency of 100 Hz.

Each of the implantable devices may transmit a response magnetic field to the base station 120. The response magnetic field can be generated by each of the implantable devices oscillating at a resonant frequency of the implantable device. The implantable devices can emit electrical signals with different timings or frequencies such that the signals constructively interfere to stimulate the target region. The interference may be created by an overlap between alternating electric fields at the same frequency produced by the multiple implantable devices. Alternatively, the interference may be created by the overlap between alternating electric fields of different frequencies produced by the multiple implantable devices.

In some instances, each implantable device may be controlled by a separate base station. Alternatively, each implantable device may be associated with a unique digital identifier that the base station 120 can use to communicate with the desired implantable device. The unique digital identifier can be a bit sequence in front of a signal. So, the base station 120 can send instructions for the different implant stimulation protocols to the implantable device 110 and additional implantable devices with the appropriate digital identifiers. As an example, the base station 120 can send instructions of the implant stimulation protocol for the implantable device 110 with the unique digital identifier of the implantable device 110. The base station 120 can also send instructions of other implant stimulation protocols for other implantable devices with other digital identifiers. Each of the implantable devices may receive each of the instructions, but may only process and execute the instructions with their corresponding digital identifier. The interference may be produced by controlling a relative timing of the electrical field produced by the implantable devices. So, the implantable device 110 may be instructed to deliver stimulation 10 ms from receiving the instructions and another implantable device may be instructed to deliver stimulation 20 ms from receiving the instructions.

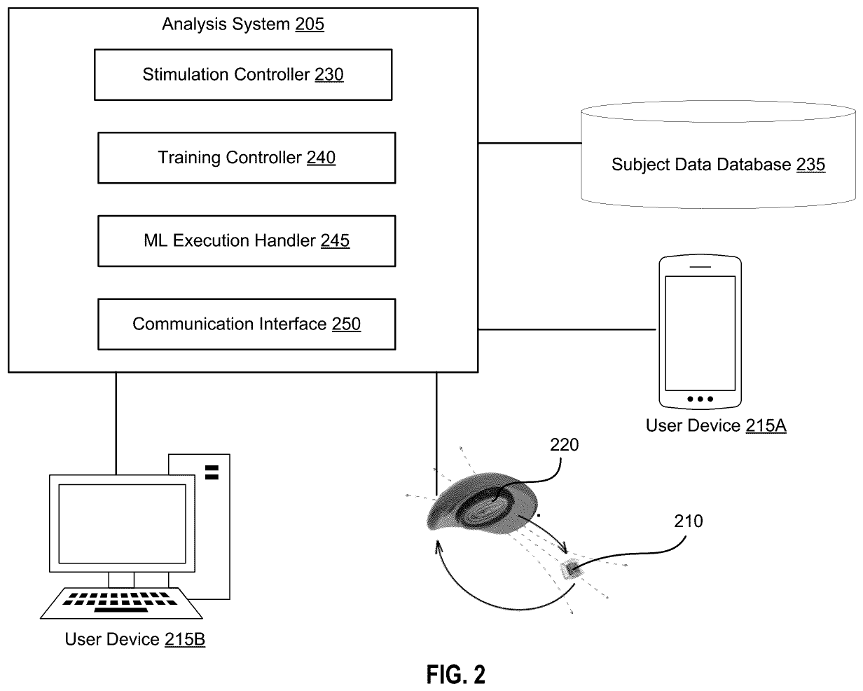

FIG. 2 illustrates an exemplary computing environment 200 for evaluation and treatment recommendation using implantable devices magnetoelectric devices according to some aspects of the present invention. The computing environment 200 can include an analysis system 205 to evaluate and recommend treatment stimulation parameters for an implantable device 210. The implantable device 210 can be in communication with a base station 220 that is in communication with the analysis system 205 and/or one or more user devices 215A-B. In an example, user device 215A may be a smart phone or other device associated with the subject in which the implantable device 210 is implanted and the user device 215B may be a desktop computer or other device associated with a care provider of the subject.

In some instances, a stimulation controller 230 can execute code to trigger delivery of stimulation by the implantable device 210. The stimulation controller 230 may trigger the stimulation based on subject data of the subject. For instance, the stimulation controller 230 can access a subject data database 235 that stores the subject data for the subject and additional subject data for other subjects. The subject data can include (for example) demographic data, medical history data, sleep data, physical activity data, physiological data, and/or mood data for the subject. The subject data may be collected based on observations or procedures performed by the care provider or by one or more sensors (e.g., a smart watch, a smart ring, etc.) worn by the subject.

The stimulation controller 230 may analyze the subject data and predict that the subject has a condition. For instance, particular medical history data, sleep data, and mood data may indicate that the subject has depression. Based on predicting that the subject has the condition, the stimulation controller 230 may select or generate software code for execution by the user device 215A and the implantable device 210. First software code can be selected that causes a presentation at a user interface of the user device 215A and second software code can be selected that includes various implant stimulation protocols. The presentation may be requests for input from the subject that are informative of an instantaneous user characteristic of the subject. Examples of the instantaneous user characteristic may be a physical or psychological attribute of the subject, such as a mood, anxiety level, or tremor severity of the subject. The requests may involve a task that is to be performed by the subject using the user device 215A while various implant stimulation protocols are delivered by the implantable device 210. The software code that is selected can depend on the predicted condition. So, the requests for input associated with a predicted condition of depression may be different than the requests for input that is associated with a predicted condition of anxiety. Alternatively, the stimulation controller 230 may receive an indicator of the predicted condition for the subject from the user device 215B upon the care provider analyzing the subject data and determining the predicted condition for the subject. The stimulation controller 230 may also receive an indication of the software code that is to be selected from the user device 215B.

The stimulation controller 230 can then send the first software code to the user device 215A and the second software code to the base station 220. Or, if the stimulation controller 230 selected or generated the software code that is to be executed, the stimulation controller 230 may send an indication of the software code to the user device 215B for approval by the care provider before the software code is sent to the user device 215A and the base station 220. In an example, the first software code may present requests for input associated with a gambling task at the user interface of the user device 215A. The gambling task may be informative of impulse control if the subject is predicted to have depression. While the subject performs the provides inputs for the gambling task using the user device 215A, the implantable device 210 can deliver the various implant stimulation protocols. The base station 220 can send a signal to the implantable device 210 with an instruction to deliver stimulation according to the various implant stimulation protocols. As the subject provides input associated with the presentation at the user device 215A and the implantable device 210 delivers stimulation, the analysis system 205 can receive one or more communications from the user device 215A, the base station 220, and/or the implantable device 210.

In some instances, the second software code may include one or more initial implant stimulation protocols, and then a machine-learning model can be executed to determine subsequent implant stimulation protocols that are to be performed during the presentation at the user device 215A and/or to determine whether an implant stimulation protocol is resulting in a target effect for the instantaneous user characteristic. Examples of the machine-learning model include a gradient boosting model, a principal component analysis model, and/or logistic regression model. The training data may include subject data obtained from the subject data database 235. The training data can also include data identifying conditions of subjects, effective implant stimulation protocols for the subjects, and/or target effects.

Training controller 240 can use the training data to train a machine-learning model. More specifically, training controller 240 can access an architecture of a model (e.g., gradient boost model), define (fixed) hyperparameters for the model (which are parameters that influence the learning rate, size, and complexity of the model, etc.), and train the model such that a set of parameters are learned. The set of parameters may be learned by identifying parameter values that are associated with a low or lowest loss, cost or error generated by comparing predicted outputs (obtained using given parameter values) with actual outputs.

A machine learning (ML) execution handler 245 can use the architecture and learned parameters to process non-training data and generate a result. For example, ML execution handler 245 may access an input data set that includes the communication(s) during at least a period of the execution of the first software code and the second software code. A communication may be generated by the user device 215A that represent inputs detected at the user interface of the user device 215A while or after the implant stimulation protocols were executed. Another communication may be generated by the base station 220 recording stimulation times at which the implantable device 210 delivers stimulation. An additional communication can include a physiological signal collected by sensors (e.g., sensors 130 in FIG. 1) physically or wirelessly connected to the base station 220. The sensors can be electrical sensors or optical sensors. For instance, the sensors may be EEG electrodes or optodes (e.g., functional near-infrared spectroscopy (fNIRS) sensors). The physiological signal collected can be based on the type of sensors. For instance, EEG electrodes may measure electrical activity (e.g., beta-band frequency) and fNIRS sensors may measure changes in blood flow or oxygenation. The input data set may include a signal generated by the implantable device 210 based on backscattering of the magnetic field. Other communications may be received from an eye tracking device and/or a facial recognition device that can detect facial expressions of the subject during the execution of the first software code and the second software code.

In some instances, multiple implantable devices are implanted into the subject and deliver stimulation during the execution of the first software code and the second software code. Each of the implantable devices may deliver the same or different stimulation to the target regions. The communications can include data collected from each of the implantable devices during the execution of the second software code. Backscattered signals generated by the implantable device 210 and additional implantable devices may include the unique digital identifiers of the implantable devices. As such, the base station 220 can de-multiplex backscattered signals received from each of the implantable devices.

In some instances, the machine-learning model can output a modification for the second software code based on the input data set. For instance, the modification may be a new implant stimulation protocol that is to be delivered by the implantable device 210 during the presentation at the user interface. As an example, the machine-learning model may determine, based on the communication(s), that the implant stimulation protocols executed previously during the presentation are not resulting in a target effect (e.g., impulsivity and/or mood are not improved for depression, hand tremors are not improved for Parkinson's, etc.). So, the machine-learning model may output a new implant stimulation protocol with a different amplitude, pulse width, frequency, shape, etc. compared to the previous implant stimulation protocols. The analysis system 205 can then modify the second software code to include the new implant stimulation protocol, and the base station 220 can cause the implantable device 210 to deliver stimulation according to the new implant stimulation protocol. The analysis system 205 can then receive new communications corresponding to the new implant stimulation protocol and determine if additional modifications should be made.

The modification may alternatively be a change to which of the implantable devices deliver stimulation during the execution of the first software code and the second software code. For instance, the machine-learning model may determine, based on the communication(s), that of three implantable devices implanted into the subject, two of the implantable devices are associated with delivering stimulation that results in a target effect for the instantaneous user characteristic. So, the machine-learning model may output an indication of a subset including the two implantable devices. The analysis system 205 can modify the second software code to cause the subset to delivery stimulation while the remaining implantable device is inactive and does not deliver stimulation. The analysis system 205 can send the modified second software code to the base station 220, which causes the subset of implantable devices to deliver stimulation. The analysis system 205 can then receive additional communications and determine if additional modifications should be made.

Subsequent to the first software code and the second software code being executed, the analysis system 205 can determine a treatment recommendation for the subject based on at least the communication from the user device 215A. The treatment recommendation may be an implant stimulation protocol that includes an amplitude (e.g., between 0.2 and 3.5 V), shape (e.g., bi-phasic or mono-phasic), pulse width (e.g., between 0.05 and 1.5 ms), and frequency (e.g., between 0 and 200 Hz) for the stimulation. In some instances, the treatment recommendation may be a modification to a current stimulation protocol that is delivered to the subject. For example, the treatment recommendation may be a modification to an amplitude, a frequency, and/or a timing of stimulation delivered by the implantable device 210. The treatment recommendation may additionally be determined based on additional communications received from the base station 220, the implantable device 210, the sensors connected to the base station 220, the eye tracking device, and/or the facial recognition device. As an example, the implantable device 210 may record neural activity during or after delivering the stimulation and the analysis system 205 can receive the recorded neural activity to determine the treatment recommendation. In some instances, the communication(s) can be input into the machine-learning model or another machine-learning model, and the machine-learning model can output a result indicating the treatment recommendation. The recommendation may be based on the implant stimulation protocols that were performed during the execution of the software code.

Similar to determining modifications for the second software code, the treatment recommendation may be determined based on a prediction of whether or a degree to which the implant stimulation protocols result in the target effect for the instantaneous user characteristic. The treatment recommendation may be based on the implant stimulation protocols being used while the input(s) were received or that preceded the receival of the input. For instance, if the communication(s) indicate a particular implant stimulation protocol during the execution of the software code resulted in the target effect, then the treatment recommendation can include a similar implant stimulation protocol. Or, if the communication(s) indicate that a subset of the implantable devices are associated with delivering stimulation that results in the target effect, then the treatment recommendation can be generated to cause the subset of implantable devices to delivery stimulation while remaining implantable devices are inactive.

A communication interface 250 can collect results and communicate the result(s) (or a processed version thereof) to the user device 215B (e.g., associated with care provider of the subject) or another system. For example, communication interface 250 may generate and output an indication of the treatment recommendation. The recommendation may then be presented and/or transmitted, which may facilitate a display of the recommended treatment, for example on a display of a computing device.

### III. Exemplary Process

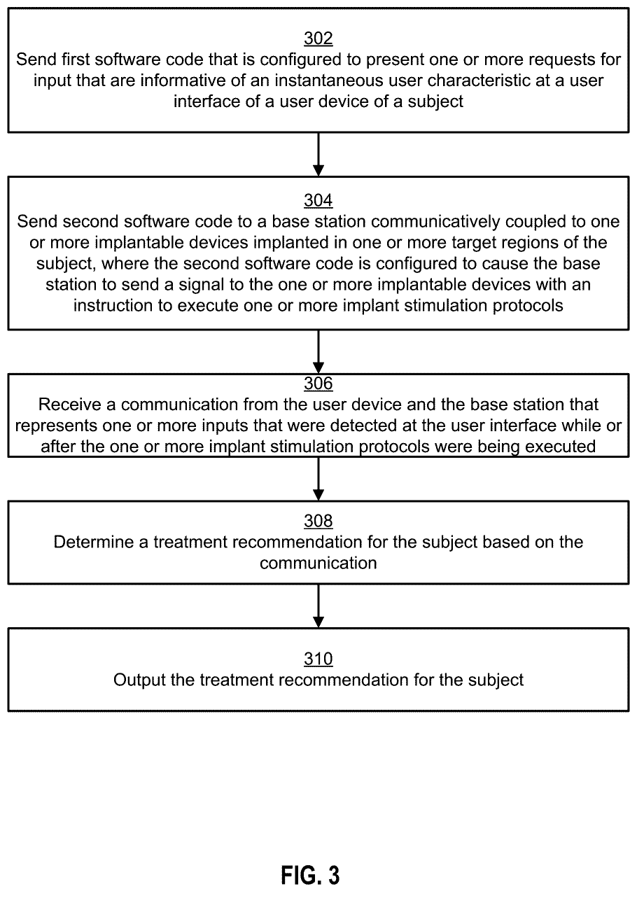

FIG. 3 illustrates a block diagram of a process for treatment evaluation and recommendation using implantable magnetoelectric devices according to some aspects of the present invention. An analysis system, such as analysis system 205 in FIG. 2, may perform the steps of FIG. 3.

At block 302, first software code is sent to a user device of a subject. The first software code can cause one or more requests for input to be presented at a user interface of the user device. The requests for input can be informative of an instantaneous user characteristic of the subject. The first software code may be selected based on subject data of the subject. The subject data can include sleep data, physical activity data, physiological data, and/or mood data collected by sensors worn by the subject or obtained based on observations performed by a care provider. In addition, the subject data may include demographic information or medical history information for the subject. Based on the subject data, the subject may be predicted to have a condition. For example, physiological data collected by an accelerometer worn by the subject indicating that the subject frequently experiences hand tremors may be an indicator of the subject having Parkinson's. The prediction of the condition may be performed by a care provider, and the analysis system can receive the indicator of the predicted condition by communicating with a provider system of the care provider. For instance, the provider system may send the indicator of the predicted condition to the analysis system based on an input command received from the care provider. The first software code can then be selected from a set of software codes that are associated with various predicted conditions. So, the first software code can be selected for the predicted condition of Parkinson's to evaluate the instantaneous user characteristic of the severity of hand tremors of the subject. As an example, the first software code may cause a presentation of a task that involves of the subject holding the user device and manipulating the user device to navigate a maze.

At block 304, second software code is sent to a base station communicatively coupled to one or more implantable devices implanted into target regions of the subject. The base station can include a magnetic field generator and a magnetic transceiver for implementing magnetoelectric communication with the one or more implantable devices. Each implantable device can include a magnetoelectric film, an electrical circuit coupled to the magnetoelectric film, and one or more electrodes. The second software code can cause the base station to send a signal to the implantable devices with an instruction to execute one or more implant stimulation protocols. The implant stimulation protocols may affect the instantaneous user characteristic of the subject during the presentation at the user interface. The second software code can be selected based on the predicted condition.

In an example, an initial implant stimulation protocol of the second software code may be a default protocol with predefined values for the amplitude, frequency, shape, pulse width that is to be delivered by each of the implantable devices. The base station can send instructions about the implant stimulation protocol to the appropriate implantable device(s). As the subject provides input at the user interface in associated with the first software code, the implantable devices deliver stimulation according to implant stimulation protocols upon receiving the instructions.

At block 306, a communication is received from the user device and the base station. The communication represents one or more inputs that were detected at the user interface while or after the one or more implant stimulation protocols were executed. The communication from the user device may be information about the input received at the user interface, such as a score or other indicator of how well the subject performs a presented task. The communication may additionally include an indicator of how the instantaneous user characteristic is affected during the execution of the implant stimulation protocols. An additional communication from the base station can be generated by the base station recording one or more stimulation times at which the implantable device delivers the stimulation to the target tissue. The one or more stimulation times can be indicated in the implant stimulation protocol. So, based on the instructions sent to the implantable device, the base station can record the stimulation times. A communication may also be received from one or more sensors that are connected to the base station or from the implantable device(s). The base station can access a physiological signal collected by the sensors, and the physiological signal can be the communication. The sensors may be EEG electrodes or optodes. The sensors can be part of a wearable component (e.g., worn around a head, neck, or waist of the user) that also includes the base station. The sensors can continuously or periodically generate the physiological signal while the base station is proximate the implantable devices. In some instances, the implantable device(s) may act as the sensors that collects the physiological signal. In addition, a communication may be received from an eye tracking device or a facial recognition device during the execution of the first software code and the second software code.

In some instances, the analysis system may predict, based on the communication(s), whether or a degree to which the implant stimulation protocols are resulting in a target effect for the instantaneous user characteristic. The communication(s) may be input into a machine-learning model that is trained to predict whether the implant stimulation protocols are resulting in the target effect or to predict stimulation parameters for an implant stimulation protocol that will result in the target effect. As an example, if the communication(s) indicate that the instantaneous user characteristic improves or that the subject has improved performance on the presented task during implant stimulation protocols with a particular frequency, the analysis system may determine that the implant stimulation protocols with the particular frequency result in the target effect. So, the second software code can be modified so that any subsequent implant stimulation protocols that are performed during the execution of the first software code include the particular frequency. In the Parkinson's example, the improved performance of navigating the maze may indicate a reduction in hand tremors when implant stimulation protocols with the particular frequency are performed. So, modifying the second software code to include only implant stimulation protocols with the particular frequency can allow additional stimulation parameters that impact the severity of hand tremors to also be determined.

The analysis system may additionally or alternatively modify the second software code based on a determination of which implantable devices deliver stimulation that results in the target effect for the instantaneous user characteristic. The prediction based on the communication(s) may indicate a subset of the implantable devices that are associated with delivering stimulation that results in the target effect, so the second software code can be modified to cause the subset of the implantable devices to deliver stimulation while remaining implantable devices are inactive during a remaining portion of the execution of the first software code. For example, the subject may have an implantable device in their motor cortex and an implantable device in their bilateral subthalamic nucleus. The machine-learning model may predict, based on the communication(s), that the stimulation delivered by the implantable device in the motor cortex results in the target effect of reduced hand tremors during the presentation at the user interface, while the stimulation delivered by the implantable device in the bilateral subthalamic nucleus does not result in the target effect. So, the second software code can be modified so that subsequent implant stimulation protocols cause the implantable device in the motor cortex to deliver stimulation while the implantable device in the bilateral subthalamic nucleus remains inactive.

At block 308, a treatment recommendation for the subject is determined based on the communication from the user device. The treatment recommendation can be an implant stimulation protocol that is delivered to the subject outside of the period in which the first software code and the second software code are executed. The treatment recommendation may be implemented to treat the predicted condition of the subject. The treatment recommendation may include a modification to an amplitude, a frequency, and/or a timing of stimulation delivered by the one or more implantable devices. To determine the treatment recommendation, similar to determining a modification to the second software code, the analysis system may predict, based on one or more communications, whether or a degree to which the implant stimulation protocols are resulting in a target effect for the instantaneous user characteristic. The communication(s) and subject data may be input into a machine-learning model that is trained to predict whether the implant stimulation protocols are resulting in the target effect or to predict stimulation parameters for an implant stimulation protocol that will result in the target effect. As an example, if the communications indicates that the subject has improved performance on a task presented at the user device during implant stimulation protocols with a particular frequency, the analysis system may determine that the implant stimulation protocols with the particular frequency result in the target effect. So, the treatment recommendation can be an implant stimulation protocol that includes the particular frequency.

The analysis system may additionally or alternatively determine the treatment recommendation based on a determination of which implantable devices deliver stimulation that results in the target effect for the instantaneous user characteristic. The prediction based on the communications (and optionally the subject data) may indicate a subset of the implantable devices that are associated with delivering stimulation that results in the target effect, so the treatment recommendation can be associated with causing the subset of the implantable devices to deliver stimulation while remaining implantable devices are inactive.

At block 310, the treatment recommendation for the subject is output. The recommendation may be output to the provider system associated with the care provider of the subject such that the care provider can approve the recommended treatment for the subject. Upon approval, the implant stimulation protocol of the treatment recommendation can be performed for the subject such that stimulation is delivered to the subject in accordance with the recommendation.

The treatment recommendation can include one or more stimulation parameters, one or more stimulation patterns, and/or a partial or full definition of a stimulation schedule. Exemplary stimulation parameters include a stimulation frequency (that identifies how often electrical pulses are delivered per unit of time during a stimulation session), a pulse width (that defines a duration of each electrical pulse), a duration of a stimulation session, a pulse amplitude (that identifies an intensity of one or more electrical pulses), burst parameter (e.g., that identifies a burst duration, interburst interval and/or a number of electrical pulses per burst), ramp-up/down time (that indicates how quickly stimulation ramps up/down in intensity at a beginning/end of a stimulation session) and/or a duty cycle (e.g., that identifies a percentage of time that stimulation is active versus off during a stimulation session). A stimulation parameter may specify a programming mode of a device (e.g., constant stimulation, cyclic stimulation, or adaptive stimulation).

A partial or full definition of a stimulation schedule may identify when and/or how frequently stimulation sessions are to occur and/or a duration of a stimulation session. For example, start times for stimulation sessions can be identified. Some stimulation treatments are configured such that initiation of stimulation sessions and/or parameter(s) of one or more stimulation sessions are dynamically determined based on a real-time or recent circumstance that relates to a state (e.g., a physical or emotional state) of a subject and/or an environmental data point. The treatment recommendation can define part or all of the real-time or recent circumstance and/or can define how the part or all of the real-time recent circumstance is to affect a stimulation aspect (e.g., when to initiate a next stimulation session, a parameter of a next stimulation session, etc.). For example, the treatment recommendation can identify on which variable(s) a stimulation aspect is to depend on, how a stimulation aspect is to depend on a given variable, etc. As one illustration, a treatment recommendation may identify one or more thresholds of a score generated based on a survey that is to be periodically completed via an electronic device by a subject, where a decision as to whether to initiate (e.g., immediately or at a defined future time) treatment or when to initiate treatment is based on whether a recent score for the subject passed a given threshold of the one or more thresholds.

FIG. 3 shows one exemplary process for controlling and monitoring effects of stimulation delivered by an implantable device. Other examples can include more steps, fewer steps, different steps, or a different order of steps.

### IV. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A method comprising:
sending, to a user device of a subject, first software code that is configured to present, at a user interface of the user device, one or more requests for input that are informative of an instantaneous user characteristic of the subject;
sending, to a base station that is communicatively coupled to one or more implantable devices implanted in one or more regions of the subject, second software code that is configured to cause the base station to send a signal to the one or more implantable devices with an instruction to execute one or more implant stimulation protocols;
receiving, from the user device and the base station, a communication that represents one or more inputs, wherein the one or more inputs were detected at the user interface while or after the one or more implant stimulation protocols were being executed;
determining a treatment recommendation for the subject based on the communication; and
outputting the treatment recommendation for the subject.

2. The method of claim 1, wherein at least one of the one or more implantable devices includes:
a magnetoelectric film;
an electrical circuit coupled to the magnetoelectric film; and
one or more electrodes.

3. The method of claim 1, wherein the base station includes:
a magnetic field generator; and
a magnetic transceiver.

4. The method of claim 1, further comprising:
receiving an additional communication from one or more sensors that are physically or wirelessly connected to the base station, or from the one or more implantable devices during the execution of the first software code and the second software code; and
determining the treatment recommendation for the subject based on the additional communication.

5. The method of claim 1, further comprising:
generating, by the base station, an additional communication by recording one or more stimulation times at which the one or more implantable devices deliver stimulation to the one or more regions;
predicting, based on the communication and the additional communication, whether or a degree to which the one or more implant stimulation protocols are resulting in a target effect for the instantaneous user characteristic; and
modifying the second software code based on the prediction.

6. The method of claim 5, wherein modifying the second software code includes:
determining a subset of the one or more implantable devices that are associated with delivering stimulation that results in the target effect; and
modifying the second software code to cause the subset of the one or more implantable devices to deliver stimulation while remaining implantable devices are inactive.

7. The method of claim 1, further comprising:
generating, by the base station, an additional communication by recording one or more stimulation times at which the one or more implantable devices deliver stimulation to the one or more regions;
predicting, based on the communication and the additional communication, whether or a degree to which the one or more implant stimulation protocols are resulting in a target effect for the instantaneous user characteristic; and
determining the treatment recommendation based on the prediction.

8. The method of claim 1, wherein determining the treatment recommendation includes:
generating, by the base station, an additional communication by recording one or more stimulation times at which the one or more implantable devices deliver stimulation to the one or more regions;
determining, based on the communication and the additional communication, a subset of the one or more implantable devices that are associated with delivering stimulation that results in a target effect for the instantaneous user characteristic; and
generating the treatment recommendation to cause the subset of the one or more implantable devices to deliver stimulation while remaining implantable devices are inactive.

9. The method of claim 1, further comprising:
accessing subject data of the subject;
determining a predicted condition for the subject based on the subject data; and
selecting the first software code and the second software code based on the predicted condition.

10. The method of claim 1, further comprising:
receiving an additional communication from an eye tracking device and/or a facial recognition device during the execution of the first software code and the second software code; and
determining the treatment recommendation for the subject based on the additional communication.

11. The method of claim 1, wherein determining the treatment recommendation includes inputting the communication into a machine-learning model.

12. The method of claim 1, wherein the treatment recommendation comprises a modification to an amplitude, a frequency, and/or a timing of stimulation delivered by the one or more implantable devices.

13. A system comprising:
one or more data processors; and
a non-transitory computer readable storage medium containing instructions which, when executed on the one or more data processors, cause the one or more data processors to perform a set of actions including:
sending, to a user device of a subject, first software code that is configured to present, at a user interface of the user device, one or more requests for input that are informative of an instantaneous user characteristic of the subject;
sending, to a base station that is communicatively coupled to one or more implantable devices implanted in one or more regions of the subject, second software code that is configured to cause the base station to send a signal to the one or more implantable devices with an instruction to execute one or more implant stimulation protocols;
receiving, from the user device and the base station, a communication that represents one or more inputs, wherein the one or more inputs were detected at the user interface while or after the one or more implant stimulation protocols were being executed;
determining a treatment recommendation for the subject based on the communication; and
outputting the treatment recommendation for the subject.

14. The system of claim 13, wherein the one or more implantable devices each include:
a magnetoelectric film;
an electrical circuit coupled to the magnetoelectric film; and
one or more electrodes.

15. The system of claim 13, wherein the base station includes:
a magnetic field generator; and
a magnetic transceiver.

16. The system of claim 13, wherein the set of actions further include:
receiving an additional communication from one or more sensors that are physically or wirelessly connected to the base station, or from the one or more implantable devices during the execution of the first software code and the second software code; and
determining the treatment recommendation for the subject based on the additional communication.

17. The system of claim 13, wherein the set of actions further include:
generating, by the base station, an additional communication by recording one or more stimulation times at which the one or more implantable devices deliver stimulation to the one or more regions;
predicting, based on the communication and the additional communication, whether or a degree to which the one or more implant stimulation protocols are resulting in a target effect for the instantaneous user characteristic; and
modifying the second software code based on the prediction.

18. The system of claim 17, wherein modifying the second software code includes:
determining a subset of the one or more implantable devices that are associated with delivering stimulation that results in the target effect; and
modifying the second software code to cause the subset of the one or more implantable devices to deliver stimulation while remaining implantable devices are inactive.

19. The system of claim 13, wherein the set of actions further include:
generating, by the base station, an additional communication by recording one or more stimulation times at which the one or more implantable devices deliver stimulation to the one or more regions;
predicting, based on the communication and the additional communication, whether or a degree to which the one or more implant stimulation protocols are resulting in a target effect for the instantaneous user characteristic; and
determining the treatment recommendation based on the prediction.

20. The system of claim 13, wherein determining the treatment recommendation includes:
generating, by the base station, an additional communication by recording one or more stimulation times at which the one or more implantable devices deliver stimulation to the one or more regions;
determining, based on the communication and the additional communication, a subset of the one or more implantable devices that are associated with delivering stimulation that results in a target effect for the instantaneous user characteristic; and
generating the treatment recommendation to cause the subset of the one or more implantable devices to deliver stimulation while remaining implantable devices are inactive.

21. The system of claim 13, wherein the set of actions further include:
accessing subject data of the subject;
determining a predicted condition for the subject based on the subject data; and
selecting the first software code and the second software code based on the predicted condition.

22. The system of claim 13, wherein the set of actions further include:
receiving an additional communication from an eye tracking device and/or a facial recognition device during the execution of the first software code and the second software code; and
determining the treatment recommendation for the subject based on the additional communication.

23. The system of claim 13, wherein determining the treatment recommendation includes inputting the communication into a machine-learning model.

24. The system of claim 13, wherein the treatment recommendation comprises a modification to an amplitude, a frequency, and/or a timing of stimulation delivered by the one or more implantable devices.

25. A computer-program product is provided that is tangibly embodied in a non-transitory machine-readable storage medium that includes instructions configured to cause one or more data processors to perform a set of actions including:
sending, to a user device of a subject, first software code that is configured to present, at a user interface of the user device, one or more requests for input that are informative of an instantaneous user characteristic of the subject;
sending, to a base station that is communicatively coupled to one or more implantable devices implanted in one or more regions of the subject, second software code that is configured to cause the base station to send a signal to the one or more implantable devices with an instruction to execute one or more implant stimulation protocols;
receiving, from the user device and the base station, a communication that represents one or more inputs, wherein the one or more inputs were detected at the user interface while or after the one or more implant stimulation protocols were being executed;
determining a treatment recommendation for the subject based on the communication; and
outputting the treatment recommendation for the subject.
